(19) United States
(12) Patent Application Publication
(10) Pub. No.: US 2026/0215867 A1
(43) Pub. Date: Jul. 30, 2026

(54) TREATMENT SYSTEM TO AUTOMATICALLY CUT AND COAGULATE TISSUE AND METHOD FOR USE

(71) Applicant: Nawei Intelligent Medical Technology (Jinan) Ltd., San Jose, CA (US)

(72) Inventors: Xuemei Lin, San Jose, CA (US); Robert R. Burnside, Mountain View, CA (US); Jeffery R. Yang, Cupertino, CA (US)

(21) Appl. No.: 19/443,240

(22) Filed: Jan. 8, 2026

Related U.S. Application Data
(63) Continuation of application No. PCT/US25/44370, filed on Aug. 30, 2025.
(60) Provisional application No. 63/689,753, filed on Sep. 1, 2024.
(60) Provisional application No. 63/689,754, filed on Sep. 1, 2024.
(60) Provisional application No. 63/689,759, filed on Sep. 1, 2024.
(60) Provisional application No. 63/742,784, filed on Jan. 7, 2025.

(51) Int. Cl. A61B 34/32; A61B 34/00; A61B 34/10; A61B 34/20; A61B 34/30; A61B 90/00

(52) U.S. Cl. CPC A61B 34/32; A61B 2034/107; A61B 2034/2063; A61B 2034/252; A61B 2034/303; A61B 2090/364; A61B 2090/378

(57) ABSTRACT

This invention provides a novel medical device for performing precise urological procedures, specifically focusing on tissue cutting and coagulation. The Image Guided Cut and Coagulate Robot leverages multiple subsystems to autonomously cut and coagulate tissues while using real-time image guidance for precise targeting and minimal collateral damage. This system is particularly suited for use in urology, though its applications can extend to other surgical fields. A significant benefit of this invention lies in its ability to provide enhanced accuracy, reducing the potential for human error, while integrating multiple processes like tissue cutting, coagulation, and cooling to protect surrounding tissues. The system incorporates a cooling circuit that avoids excess heating of the surgical region.

### RELATED APPLICATION

This application is a Bypass Continuation of International Application Serial No. PCT/US25/44370, entitled TREATMENT SYSTEM TO AUTOMATICALLY CUT AND COAGULATE TISSUE AND METHOD FOR USE, filed Aug. 30, 2025, which claims the benefit of co-pending U.S. Provisional Application Ser. No. 63/742,784, entitled TREATMENT SYSTEM TO AUTOMATICALLY CUT AND COAGULATE TISSUE AND METHOD FOR USE, filed Jan. 7, 2025 and co-pending U.S. Provisional Application Ser. No. 63/689,753, entitled A TREATMENT SYSTEM TO AUTOMATICALLY CUT AND COAGULATE TISSUE, filed Sep. 1, 2024, and co-pending U.S. Provisional Application Ser. No. 63/689,754, entitled AN IMAGE GUIDED UROLOGY TREATMENT SYSTEM, filed Sep. 1, 2024, and co-pending U.S. Provisional Application Ser. No. 63/689,759, entitled A TREATMENT SYSTEM TO AUTOMATICALLY COAGULATE TISSUE FOR GYNECOLOGOGY APPLICATION, filed Sep. 1, 2024, the teachings of each of which applications are expressly incorporated herein by reference.

### FIELD OF THE INVENTION

This invention relates to automated surgical and medical treatment systems and methods, and more particularly to systems used in urology.

### BACKGROUND OF THE INVENTION

The use of robotic surgical systems and devices has been increasing in popularity over recent years. Such systems can employ visual, tactile and other forms of sensors, as well as (e.g.) video, ultrasound, X-ray, CT, MRI, etc. imaging to navigate the patient's anatomy. In the case of visual imaging (in visible or near-visible wavelengths), the robotic manipulator can be servoed based upon pattern recognition applications. The applications can employ traditional machine vision algorithms and or deep learning/AI neural networks.

Robotic manipulators can perform a variety of surgical tasks using appropriate tools provided upon an end effector (i.e. at the distal end of the manipulator). One area in which robotic surgery is desirable is urologic applications, where speed and accuracy are significant concerns.

### SUMMARY OF THE INVENTION

This invention overcomes limitations in prior art by providing a novel medical device for performing precise urological procedures, specifically focusing on tissue cutting and coagulation. The Image Guided Cut and Coagulate Robot leverages multiple subsystems to autonomously cut and coagulate tissues while using real-time image guidance for precise targeting and minimal collateral damage. This system is particularly suited for use in urology, though its applications can extend to other surgical fields. The key benefit of this invention lies in its ability to provide enhanced accuracy, reducing the potential for human error, while integrating multiple processes like tissue cutting, coagulation, and cooling to protect surrounding tissues.

Unlike prior systems that either require manual operation or are limited to individual functions (e.g., cutting alone without coagulation), this invention offers a fully integrated approach. By combining autonomous operation with advanced imaging technology, the robot ensures that surgeons can perform complex procedures with greater precision and efficiency. The robot's ability to adjust energy output, as well as to deliver targeted cooling, makes it a valuable tool for minimizing tissue damage in sensitive surgical areas. The cooling function is essential to protect healthy tissues from thermal damage, an issue commonly encountered with high-energy surgical tools.

In an exemplary embodiment, the system comprises three main subsystems: the System Control Unit, the Delivery Unit, and the System Console. These components work in tandem to achieve real-time, image-guided tissue treatment, with feedback mechanisms ensuring accuracy and safety throughout the procedure. The system provides automated control of cutting and coagulation energy delivery, minimizing the surgeon's manual intervention and enabling a more streamlined workflow. Additionally, the system's cooling mechanism preserves the integrity of surrounding tissues, making the system safe for use in delicate areas, such as near vital organs or sensitive structures.

In an illustrative embodiment, an autonomous system (and associated method) for surgery on a region of a patient provides an imaging transducer that images the region in real time, and a processor that receives the images and recognizes features therein related to the region. A robotic treatment probe is constructed an arranged to perform surgical tissue cutting and coagulation on the region based upon instructions from the processor. An active cooling circuit that circulates predetermined volumes of fluid at the region. This cooling is controlled/modulated based upon local temperature and/or other sensed parameters. Illustratively, the system can include an aspiration circuit that directs fluid from the region to a remote location for collection. The aspiration circuit can capture fluid and enable volumetric analysis, and/or can include a filter that captures particulates for analysis. The region can be the peritoneal region, and the surgery can be a urological procedure, which is performed thereon. A graphical user interface can be constructed and arranged to enable planning of a path of the surgery and treatment probe based upon real-time images overlaid on prior-acquired images from a scanning modality. Illustratively, the imaging transducer comprises an ultrasound transducer that provides images to a user.

A method for performing urological surgery with the above-described system can consist of preparing the surgical site and locating the imaging transducer and treatment probe with respect to the region, locating a lithotomy position, beginning imaging with the imaging transducer, inserting the treatment probe and adjusting a sonogram from the imaging transducer using a user interface. The method further consists of positioning the treatment probe at a home position, developing a treatment plan on the user interface and irrigating the region. Then treatment of the region is then performed, including initiation, pulverization, aspiration, hemostasis, based upon motion control of the treatment probe based upon the processor. The processor can be adapted to operate the treatment probe autonomously, and can employ a 3D treatment plan that maps a treatment region of the patient and follow program steps to perform the method.

### BRIEF DESCRIPTION OF THE DRAWINGS

The invention description below refers to the accompanying drawings, of which:

FIG. 1 is a diagram showing an overall system for conducting robotically guided surgery for (e.g.) urologic procedures according to an illustrative embodiment;

FIG. 2 is a block diagram showing functional components of the system of FIG. 1, including three primary components thereof;

FIG. 3 is a diagram showing an exemplary GUI display of the treatment path in the arrangement of FIG. 1;

FIG. 4 is a flow diagram showing an overall operational procedure for the system and method according to the arrangement of FIG. 1;

FIG. 5 is a flow diagram showing the preparation steps in the overall procedure of FIG. 4;

FIG. 6 is a flow diagram showing the insertion and alignment steps in the overall procedure of FIG. 4;

FIG. 7 is a flow diagram showing the position and planning steps in the overall procedure of FIG. 4;

FIG. 8 is a flow diagram showing the treatment steps in the overall procedure of FIG. 4; and

FIG. 9 is a flow diagram of the post-treatment, cleanup steps in the overall procedure of FIG. 4.

### DETAILED DESCRIPTION

### I. System Overview

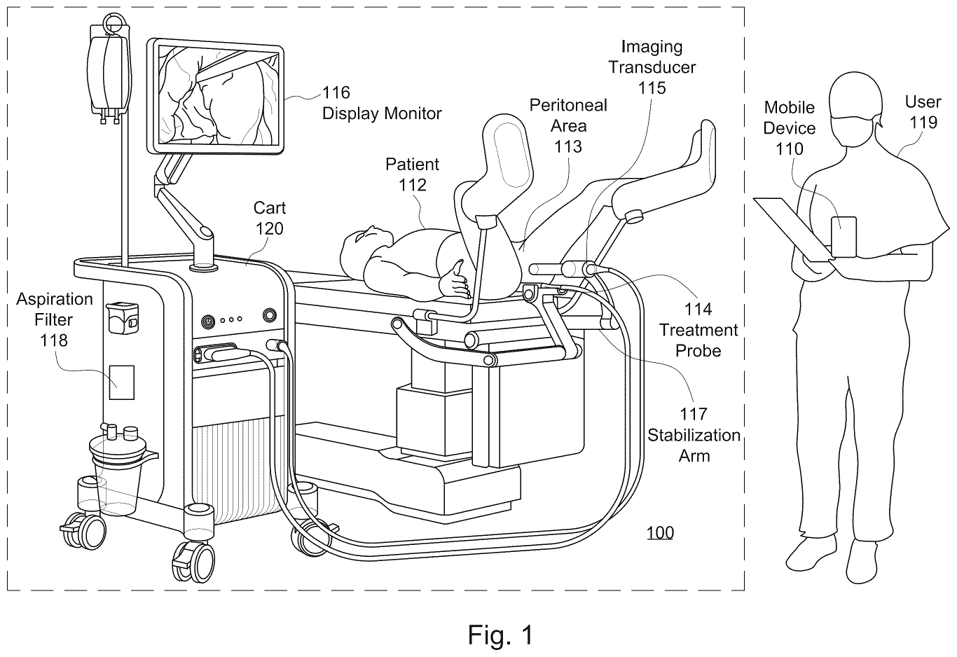

Reference is made to FIG. 1, which depicts an overall arrangement 100 of the system and method for autonomous tissue cutting and coagulation in a patient (patient's/subject's) body 112. This system and method provides a rapid and efficient surgical intervention that is obtainable in image-guided approaches according to the embodiments herein. The arrangement 100 shows the treatment probe 114 adapted to be inserted into peritoneal area 113 of the body 112 in position relative to the a commercially available, (e.g.) third party ultrasound transducer device 115 while the user (a medical practitioner 119) observes and guides the procedure on the display monitor 116. As shown, such techniques can allow for a total planning and treatment time in the range of (e.g.) 20-30 minutes, when successful. According to the novel system and method herein, the traditional surgical modalities, and associated results, can be substituted with autonomous tissue cutting and coagulation. As described below, these robotic treatment arrangements employ 3D imaging sensors such as 3D ultrasound that are typically inserted into the patient's body during the procedure. By way of a non-limiting example, following an autonomous pre-planned treatment path can effectively reduce the treatment time for (e.g.) benign prostatic hyperplasia (BPH) to less than 15 minutes.

Overall, the system and method features autonomous treatment according to an (e.g.) AI-assisted image-guided treatment plan, which advantageously increases the probability of successfully completing treatments rapidly, which likewise helps to avoid excessive bleeding, urinary incontinence, and/or sexual side effects which may otherwise be encountered with other treatment arrangements.

### II. System Details for Image Guided Cut and Coagulate Robot for Urological Procedures

### 1. General Overview of the System

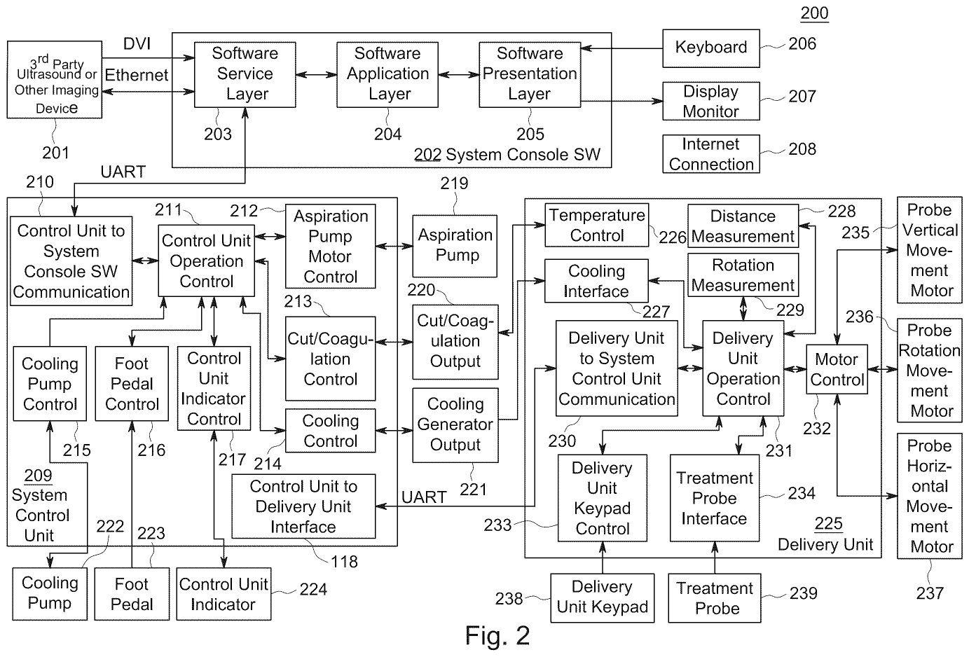

The Image Guided Cut and Coagulate Robot is designed to perform autonomous tissue cutting and coagulation, particularly in urological procedures. Reference is also made to FIG. 2, a (high-level) block diagram of the overall system 200 is shown. It depicts three primary components: the System Control Unit 209, the Delivery Unit 225, and the System Console 202. These components operate together to provide real-time image-guided treatment with precise control over energy delivery and tissue cooling. The System Control Unit 209 serves as the control center, managing the delivery of cutting and coagulation energy used by the treatment probe (distal end of the robotic manipulator), as well as cooling outputs to the Delivery Unit 225. The System Console 202, in turn, provides User Interface (UI) oversight of the entire treatment process, based upon real-time 3D imaging and immediate endoscopic visualization of the treatment area, allowing for accurate targeting of tissues during the procedure.

### 2. Application in Urological Procedures

The system is designed primarily for use in urological surgeries, where precision is critical for procedures such as tumor resection, stone removal, and treatment of benign prostatic hyperplasia (BPH). Traditional surgical tools may lack the precision required to target small, delicate areas in the urinary tract, but this invention addresses that limitation by providing accurate, real-time imaging and precise control over cutting and coagulation. The autonomous nature of the system reduces the reliance on manual intervention, thereby improving outcomes and reducing recovery times for patients.

### 3. Autonomous Operation and Image Guidance

The system's autonomous operation is a major advancement over prior technology. Through the use of real-time 3D imaging shown by the System Console 202, the system can autonomously move to the targeted treatment area and perform cutting and coagulation with minimal input from the surgeon. The user interface allows the surgeon to visualize the treatment area from multiple perspectives, offering full control over the procedure while benefiting from the system's automated functionality. This combination of automation and image guidance significantly reduces the margin of error, enhancing both safety and efficacy.

### 4. Benefits Over Existing Technology

Existing urological treatment systems often rely on separate tools for cutting and coagulation, and these tools generally require manual operation. This invention overcomes the disadvantages of traditional systems by integrating cutting, coagulation, and cooling functions into a single robotic device. Furthermore, the real-time image guidance system allows for greater precision and control, reducing the likelihood of damage to surrounding tissues. The system also offers a modular design, enabling it to be adapted for various urological and non-urological procedures, increasing its versatility and range of applications.

### 5. Subsystem Descriptions

### A. System Control Unit

With further reference to FIG. 2, the System Control Unit 209 serves as the core control hub of the system, managing the energy output for cutting, coagulation, and cooling. It monitors the status of the Delivery Unit 225 and sends real-time commands to adjust energy levels based on feedback from the System Console 202. These operational units can all be generally instantiated within the cart 120 and its interconnected peripherals.

### 1. Centralized Control

The Control Unit Operation Control 211 implements microprocessor or microcontroller-based management of System Control Unit functions, ensuring that all components work in coordination, providing seamless integration between the cutting, coagulation, and cooling processes. The Control Unit Operation Control 211 utilizes a real-time operating system to constantly monitor the status of the procedure, making real-time adjustments to energy output and cooling levels as needed.

Interfaces to other subsystems are managed by dedicated hardware included in the System Control Unit 209. For example, the System Control Unit to System Console Communication 210 is typically a type of serial port or UART connection communicating UI information. Other communication arrangements and/or protocols, which should be clear to those of skill, are also contemplated. Interface to the Aspiration Pump 219 and Cooling Pump 222 is provided by the Aspiration Pump Motor Control 212 and Cooling Pump Control 215, respectively. These allow for the surgical area and instrument(s) to be receive a flow cooling fluid to avoid overheating and heat damage.

The user can initiate on/off control of treatment using the Foot Pedal 223 (physically placed via a wired or wireless connection adjacent to the user 119), and which is operatively connected to the control unit interfaced through the Foot Pedal Control 216. The control 216 can provide a brief alphanumeric 1 or 2-line status information via the Control Unit Indicator 224, connected through Control Unit Indicator Control 217. Such controls and indicators can be implemented using commercially available and commonly utilized components in typical medical equipment arrangements.

The cutting and coagulation processes are managed through (e.g.) three (3) separate interfaces that convey commands and feedback to and from the Delivery Unit 225 to ensure precise temperature control, so that tissue is treated without causing excessive heat damage to surrounding areas. This feature allows for safe, efficient cutting while reducing the risk of unintended injury to adjacent tissues.

One of the cutting and coagulation interfaces is the Cut/Coagulation Control 213, which provides the Cut/Coagulation Output 220 to the Delivery Unit 225. Another is the Cooling Control 214, which regulates the variable amount of liquid cooling supplied to the Delivery Unit 225 by management of the Cooling Generator Output 221. Cooling can be implemented by one of many conventional methods, including thermoelectric, compressor refrigeration, etc. Additionally, the Control Unit to Delivery Unit Interface 218 is typically a type of serial port, UART, or other appropriate protocol, connection communicating command and control information between subsystems.

### B. Delivery Unit

The Delivery Unit 225 is responsible for delivering the cutting, coagulation, and cooling outputs to the treatment area. This subsystem is designed for autonomous operation, enabling it to accurately target and treat tissues based on commands from the System Control Unit 209, returning feedback on treatment progress.

Overall functioning of the Delivery Control Unit 225 is managed by the Delivery Unit Operation Control 231, which implements microprocessor or microcontroller based management of Delivery Control Unit functions.

Interface to the System Control Unit 209 is furnished by the Delivery Unit to System Control Unit Communication 230 typically implemented using a type of serial port, UART (or other appropriate protocol) connection communicating command and control information between subsystems. The other two interfaces to the System Control Unit 209 are carried out through Temperature Control hardware 226 and Cooling Interface 227.

To provide accurate motion control of the Treatment Probe 239 (also physical represented by element 114 in FIG. 1) for both position and velocity, the Delivery Unit 225 is equipped with probe motion Distance Measurement 228 and Rotation Measurement 229. These can be implemented using solid state and/or electric-mechanical components, including robot motion encoders, accelerometers, etc., in a manner clear to those of skill. The interface to the probe movement function can be a multi-axis Motor Control 232, which drives/monitors the Probe Vertical Movement Motor 235, Probe Rotation Movement Motor 236 and Probe Horizontal Movement Motor 237. The electrical interface to the Treatment Probe 239 is routed through the Treatment Probe Interface 234. Note that the probe can be driven by a variety of mechanism known to those of skill, including pneumatic/hydraulic actuators, geared drives, screw drives and/or electric stepper motors. Appropriate motion sensing and regulating mechanisms, including step counters and/or encoders can be employed to provide measure motion control and/or motion (and/or position) feedback to the robot controller.

In general, the user can interact with certain treatment parameters utilizing the Delivery Unit Keypad 238 interfaced through the Delivery Unit Keypad Control 233, which are part of the overall UI functionality.

1. Tissue Protection with Cooling Mechanism

A novel aspect of the Delivery Unit 225 is the inclusion of a cooling mechanism, which is activated to protect surrounding tissues from thermal damage. The cooling system is fully integrated into the system's treatment delivery, ensuring that the maximum temperature of the treated area is carefully managed throughout the procedure.

### 2. Autonomous Movement and Targeting

Through the use of advanced motion control, the Delivery Unit 225 can autonomously position itself in the treatment area and deliver energy with requisite pinpoint accuracy. This autonomous capability reduces the surgeon's workload and enhances the overall precision of the procedure.

### 3. Autonomous Movement and Targeting

The energy output employed for cutting and coagulation is modulated by the software in the Delivery Unit Operation Control 231 based on the specific requirements of the procedure. This ensures that the appropriate amount of energy is delivered to the tissue, minimizing the risk of overtreatment or undertreatment. The unit 231 can be regulated base upon an algorithm and/or can include a feedback loop that senses current energy output, and/or effects therefrom, and modulates output based upon the sensed level.

### 4. Intake/Output Monitoring and Control

Pumping control from fluid irrigation and aspiration results in volumetric intake/output reporting of total captured Aspiration Liquid (volume, blood%, etc.), available on the display of the Delivery Unit Keypad 238. Optionally, aspirated particulate can be captured by filter elements of the disposable aspiration set for later laboratory analysis. As also shown in FIG. 1, an Aspiration Filter Element 118 is removably installed on the cart 120 that also supports the display monitor 116, and other processing and power-supplying components of the system arrangement 100. These components are described further below.

### C. System Console

The System Console plays a significant role in the system by providing a Graphical User Interface (GUI) to real-time, 3D imaging of the treatment area, via (e.g.) a Display Monitor 207 (shown physically as monitor with image 116 in FIG. 1). This subsystem allows the user/practitioner/surgeon to visualize the targeted tissues from multiple perspectives, enabling precise control over the procedure.

The requisite software subsystems for controlling the system as provided in FIG. 2 can be implemented on commercially available desktop PC hardware, for example those employing AMD64 microprocessor architecture and/or Intel Core® processor, video capture hardware, and (e.g.) Microsoft Windows® operating system. Such software subsystems include a Software Service Layer 203 to communicate via industry-standard interfaces such as Ethernet and DVI (HDMI, etc.) to the operating console of a commercially available third party Ultrasound Device 201, such as those available from Siemens. The Software Application Layer 204 utilizes the services provided by the previous layer, and provides the GUI display and control elements to the Software Presentation Layer 205. A network connection, for example the depicted Internet Connection 208, is provided for stored medical image retrieval from previous diagnostic procedures, as well as archiving, billing, and software update purposes.

### 1. Acquisition of 3D Images

The system acquires 3D images from the operating console of the third party Ultrasound Device 201 from multiple angles of attack, which are displayed on the Display Monitor 207, which the user controls through the Keyboard 206. The keyboard can also be used to enter patient demographic information, and/or other documentation for the treatment procedure. Displaying two-dimensional (2D) views of these three-dimensional (3D) images allows the surgeon to plan and execute the procedure with enhanced accuracy, making it easier to navigate complex anatomical structures.

2. Integration with the Main Control Station

The System Console 202 is fully integrated with the System Control Unit 209 by a bidirectional communication interface, allowing for real-time adjustments based on feedback from the imaging system, typically through a serial port, UART, or other protocol connection. This integration ensures that the treatment delivery is precisely aligned with the imaging data, further enhancing the accuracy of the procedure.

### III. Treatment Techniques and Results

### A. Overall Arrangement

The system and method employs a preplanned autonomous treatment, to select the best surgical approach among several parameters before treatment starts, allowing for thorough preparation and consideration of various factors such as anatomic variation and extent of disease. In contrast, real-time manual surgical guidance offers the flexibility to adjust treatment paths based on immediate imaging feedback, and other real-time factors, ensuring a dynamic and adaptive treatment. This manual method can be desirable when a highly-skilled operator deals with unexpected obstacles or when precise timing is crucial, as it can provide increased efficiency if surgical conditions rapidly change. Notably, the visibility of critical structures through 3D real-time imaging provides a stable basis for autonomous treatment, reducing dependence upon operator skill and experience. A significant difference lies in the balance between adaptability and preparedness; real-time treatment maximizes flexibility and emphasizes operator skill and experience, while pre-planned autonomous treatment emphasizes foresight and control.

Reference is again made to FIG. 1, which shows the overall arrangement 100 of the system and method in a patient's (subject's) body 112. As noted, this arrangement allows for more rapid and repeatable surgical treatment of urological conditions that is obtainable with fewer side effects using the autonomous, image-guided approaches according to the embodiments herein.

The arrangement 100 of FIG. 1 shows the location relative to the patient's peritoneal area 113 of the treatment probe 114 and the imaging transducer 115. As shown, such techniques can allow for treatment in the range of 20-30 minutes, when successful. Overall, the system and method provides autonomous image-guided treatment which advantageously increases the probability of rapidly treating urological symptoms with a minimum of undesired side effects which may otherwise occur with other treatment modalities.

### B. Alternative Energy Sources

By way of further background, previous innovations in urological surgical intervention have fallen into two broad categories, namely, (a) mechanical approaches, or (b) energy delivery approaches. In general, such schemes/approaches have failed to perform as intended and/or else have never been widely adopted for use in the field of urology. Rotary cutters, water jets, lasers and traditional radiofrequency ablation have failed to provide a modality whose surgical performance was acceptable without undesirable clinical side effects. The illustrative system and method herein utilizes various unique techniques of energy delivery with active cooling via autonomous motion control and machine learning. Hence, the system and method can effectively address a broad range of clinical applications by including various inexpensive, clinically and commercially validated off-the-shelf technologies, such as robotics and fluid management.

### C. Enhanced Hemostasis and Recovery Times

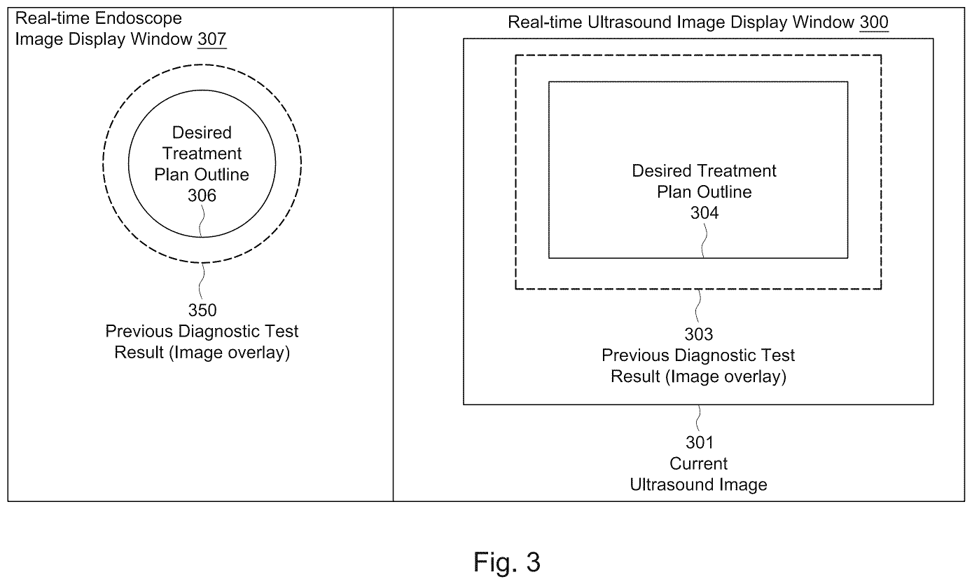

The system and method herein takes into account unwanted injury to adjacent blood vessels using (by way of non-limiting example) non-thermal electrical energy. Note that other techniques for improved hemostasis should be clear to those of skill, including, but not limited to applying active cooling, real-time limiting of electric field density, and/or various waveforms of applied electrical currents. Moreover, a common challenge in applying machine-learning to autonomous treatment is a lack of attention given towards motion control improvements that can provide more robust accuracy and precision in treatment guidance in the first place. It is contemplated that commercially available robotics technologies can be refined so that positional accuracy can be as high as possible. These challenges can include kinematic errors arising from inaccuracies in the mechanical structure, such as manufacturing tolerances and assembly errors, non-kinematic errors like temperature variations, joint compliance, and gear backlash which can significantly impact accuracy, environmental disturbances, calibration limitations, and differences in tools and materials used that can introduce inconsistencies in performance. These challenges involve inherently large patient-to-patient variations in anatomic positioning of internal organs and structures. Machine-learning techniques can be applied to address these sources of variability, allowing relatively inexperienced users to navigate treatments quickly and efficiently. By way of non-limiting example, the user-defined treatment path (304 in FIG. 3) can be further modified/enhanced by machine learning, comprising ultrasound or other real time imaging feedback during a urological treatment.

Further reference is made to FIG. 3, the treatment path user interface (UI), which shows two zones: a real-time endoscope image display window 207 and real-time ultrasound image display window. The real-time ultrasound image display window 300 (that can be part of the GUI display 116 in FIG. 1) contains the current real-time ultrasound image 301 in the background. As an overlay, a stored image 303 of a previous diagnostic test, delivered via the internet, is presented to the user. By way of non-limiting example, this may be a CT or MRI image (or another appropriate scanning modality). Using appropriate drawing controls, the user-defined treatment path 304 can be outlined by the user. The real-time endoscope image display window 307 contains the current real-time image from the treatment probe's built-in endoscopic camera in the background. As an overlay, a stored image 305 from a previous diagnostic test, delivered via the internet, is presented to the user in the same perspective as the endoscopic image. By way of non-limiting example, this image may be a CT or MRI image. The desired treatment plan outline 306 is presented as an additional overlay from the same perspective as the endoscopic image, based upon the user-defined treatment path 304 shown on the real time ultrasound image display window 308.

In the real time ultrasound image display window 308, the user may switch between different 2D representations of the 3D ultrasound scan. The image from previous diagnostic test result image 203 will indicate the location of the desired treatment area. The user can use this image to identify the boundaries for the surgical treatment area, and to draw the desired treatment plan outline 204. The system software will initially provide suggested boundaries which can be modified by the user if desired.

The system also offers a mobile application for use on a mobile device (e.g. a tablet, smartphone, laptop PC, etc. 110 (FIG. 1) to communicate with the console system. This mobile device 110 may be a cellular telephone, tablet computer, or other commercially available devices commonly used by medical users. Using the mobile device 110, the user may send the treatment plan outline to other users for consultation purposes. Any treatment plan outline changes suggested by the consulting user will be received through the internet connection (208 in FIG. 2) and displayed in the desired treatment plan outline (304 in FIG. 3). The operating user may then confirm the final treatment plan outline on the real-time ultrasound image display window 308 prior to initiating treatment.

The real-time endoscope image display window (307 in FIG. 3) is provided for display only and is not utilized for revising or confirming the treatment plan outline.

Note that 3D ultrasound is one of a variety of sensor types that can be employed to provide desired real-time spatial data. It should clear to those of skill that other types of 3D sensing devices can be employed herein. It is recognized in implementing the system and method herein that accurate and precise positioning of treatment can significantly improve the quality of the anatomic features to be used for machine learning. Such preprocessing can potentially include the use of voxel-based imaging processing and analysis. In particular, Voxel-based Nearest Neighbor (VNN) techniques can enhance the speed and accuracy of 3D reconstruction.

It is contemplated that the autonomous treatment techniques herein can further employ machine learning via, for example, 3D convolutional neural networks (CNNs). CNNs are powerful image classification tools that do not overly rely on preprocessing steps such as feature extraction and noise filtering. Note that other forms of machine learning, and/or artificial intelligence (AI) techniques and algorithms can be employed in alternate implementations as well. Learning of the AI can be based upon a library of images of normal and diseased tissue that allow for recognition of imaged feature in the patient and guidance of the robotic tip thereby. Imaging of the position of the tip with respect to the tissue can also be used to verify positioning. Additionally, the actual spatial position of the distal end of the tip can be determined by the robot's motion control and position sensing (e.g. position sensors/encoders) in 3D space. Note that the AI can be augmented by real-time learning as procedures are performed an additional images are acquired. Manual control can be recorded to supplement possible reactions by the robot to particular conditions exhibited in images. It should be clear to those of skill in the art of AI programming how such learning can be implemented.

### D. Analysis and Measurement of Aspirated Liquid Waste

Volumetric and optical analysis of aspiration liquid can be employed by the system and method. Techniques for measurement of liquid irrigation intake and aspirated waste output are well known to those skilled in the art. These techniques have been found to prevent hypervolemia and hypovolemia (and associated serious side effects) in patients undergoing surgical procedures. An associated optical measurement of relative hemoglobin concentration can be advantageously included for assessment of intraoperative blood loss.

Optionally, the aspiration screen filter element (118 in FIG. 1) can be included in the associated irrigation/aspiration tubing set for the selective collection of pulverized excised tissue, solid particulate or thrombus for subsequent analysis. Filtering elements designed to selectively capture particulate sizes of clinical interest can be removable prior to tubing set disposal.

### IV. Imaging Sensors

An image sensing device (201 in FIG. 2) is provided as part of the system and method herein. A goal of such a device is enhancing and optimizing the autonomous treatment path with a 3D perspective. Imaging can be achieved in various ways, including, but not limited to the use of 3D ultrasound and/or endoscopic optical imaging alone or fusion with prior MRI/CT, or other prior images. Where 3D ultrasound is employed, it can provide sufficient spatial resolution after performing a separate alignment step to register the 2D perspective of the 3D image (301 in FIG. 3) on the System Console UI together with a coplanar view of the treatment probe. Distance measurements at more than one physiologically distinct anatomic location thereby allows for the use of machine learning derived pattern recognition as described herein. Hence, this combination results in previously unobtainable accuracy and speed for tissue cutting and coagulation results in the 1-2 mm range after just 15-20 minutes of overall treatment.

Referring again to FIG. 1, in an exemplary implementation, the sensing system can employ (e.g.) a customized stabilization arm apparatus 117 that is readily lockable and well suited for the surgical environment. Such an easily adjusted, articulated stabilization arm can thereby provide a solid platform for robotic fine mechanical adjustment of the treatment probe position and movement trajectory. The medical support arm design can be based upon previous designs reported in the literature. The treatment probe robotic drive, with vertical, horizontal, and rotational movement can be interfaced to the medical support arm with additional customized attachment features. In an exemplary implementation by way of illustration without limitation, the medical support arm can provide (e.g.) six degrees of freedom to make it possible to target any point within a wide radius, allowing the operator to position the initial position of treatment probe exactly where a firm hold is needed. The medical support arm can provide a handle that when activated, allows guiding the arm to the desired point. As soon as the handle is released, the arm position is automatically locked.

In implementing the system and method, it is contemplated that the variation in tissue thicknesses and desired treatment area across human subjects, and along with endoscopic image acquisition of the tissue surface, can be compared to baseline values for autonomous treatment control. A 3D model can be developed and allow determination of risk to unintended treatment areas, which can be displayed to the user prior to the initiation of treatment overlaid on the medical images. This data can be employed in accordance with skill in the art to provide the appropriate path correction to the baseline treatment plan.

### V. Operation of the System and Method

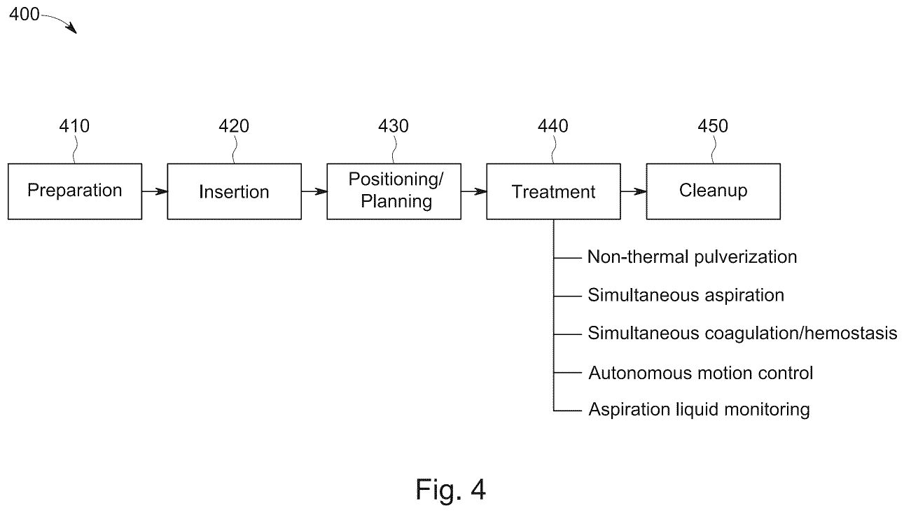

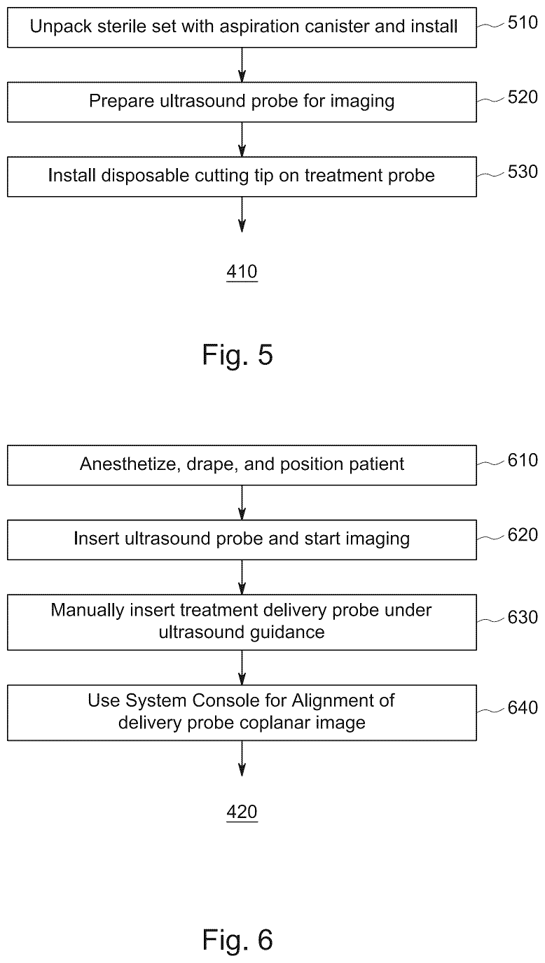

In a treatment environment with a patient, it is contemplated that the system and method can be deployed to autonomously position itself in the treatment area and deliver energy with pinpoint accuracy. An overview of this process 400 is provided in FIG. 4. The first part of the overall operational process 400 is the preparation step 410. This is further shown in FIG. 5. In step 510, before the subject enters the procedure room, operating room personnel unpack the sterile set with aspiration container and install it on the system treatment cart (120 in FIG. 1). Then, the ultrasound probe is prepared for imaging (step 520). Then, a new disposable cutting tip is affixed to the treatment probe (114 on FIG. 1) with integrated endoscopic camera (Step 530). These steps are illustrated in FIG. 5.

FIG. 6 describes patient preparation. A recipient is placed into the lithotomy position, draped and anesthetized in step 610. Then, in accordance with the overall insertion step 420 (FIG. 4), the rectal ultrasound probe (115 in FIG. 1) is inserted (step 620) and imaging is initiated through the 3rd party ultrasound console (step 630). Finally, the clinical manually inserts the treatment delivery probe under ultrasound guidance (step 640). Using the system console, a coplanar image of the treatment probe is aligned and adjusted to make ready for treatment positioning and planning (overall step 430 in FIG. 4).

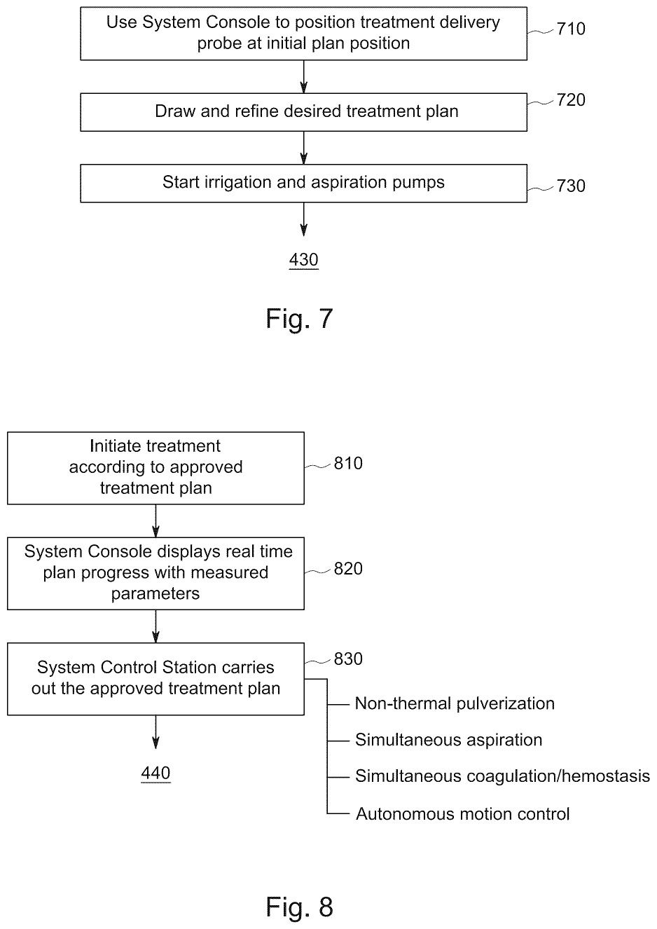

As depicted in FIG. 7, the system console is then used to command the robotic treatment (overall step 440), including probe motion control to move to the initial home location where treatment will be initiated (step 710). Then a proposed treatment plan can be drawn and refined on the screen (step 720). During this stage, the irrigation and aspiration pumps are started with a confirmation of adequate liquid flow (step 730).

Treatment (overall step 440 in FIG. 4) begins when the treatment plan is approved and the clinician starts autonomous operation of the system (step 810 in FIG. 8). During treatment, the system console displays real time plan progress with measured parameters displayed (step 820) until the plan is successfully completed (step 830).

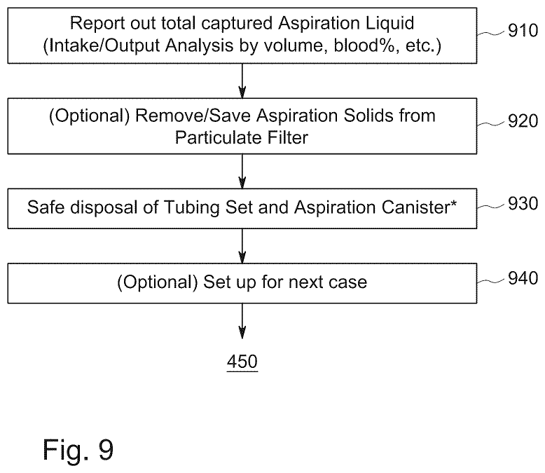

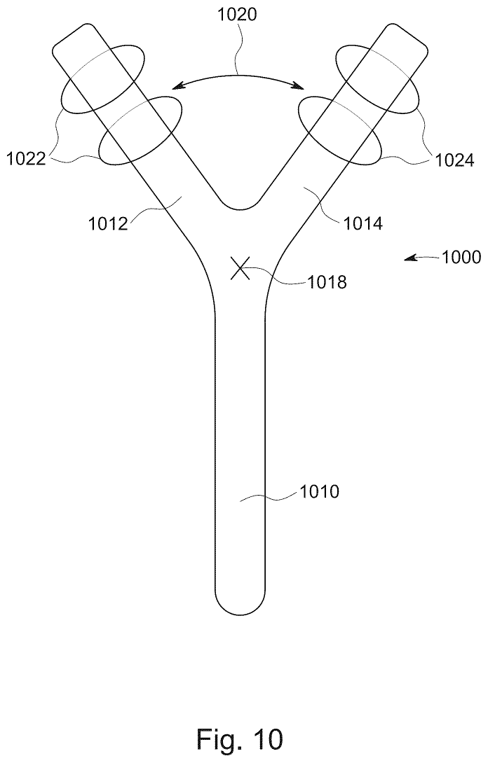

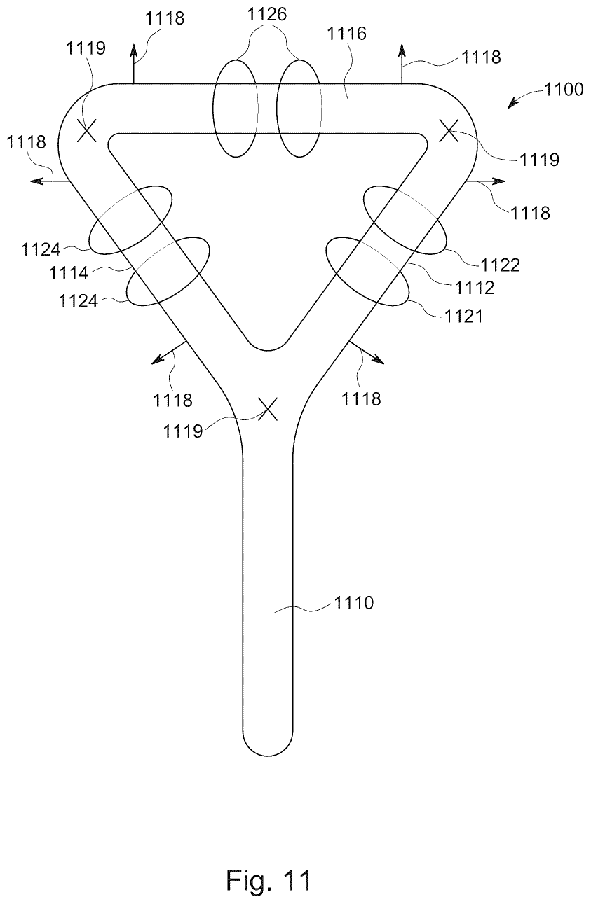

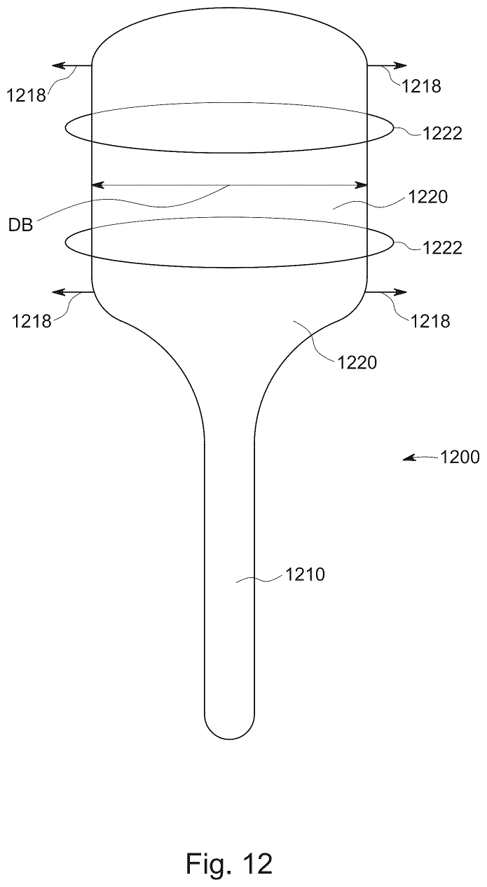

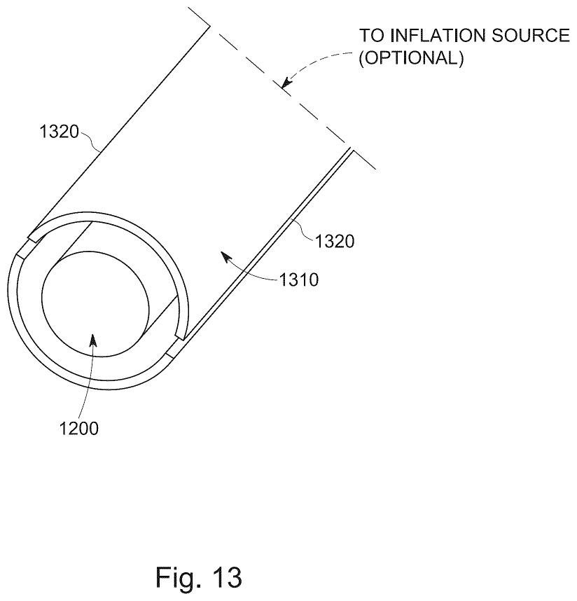

After completion, the cleanup procedure (overall step 450 in FIG. 4) occurs, as represented in FIG. 9. In step 910, the system stops pumping and reports out total captured aspiration liquid, including an intake/output analysis by volume, blood%, etc. If the optional particulate filter is utilized, it can be removed (step 920). At the end of the procedure, the entire used tubing set including the aspiration canister is disposed, ensuring no contact with biohazards (step 930). Optionally, the operating room can then be reset to prepare for the next procedure in step 940.

### VI. Autonomous Treatment

At the start of an autonomous treatment process, according to this system and method, the cooling pump (222 in FIG. 2) delivers the intake cooled saline to the treatment area through the treatment probe (114 in FIG. 1). Simultaneously, the aspiration pump (214 in FIG. 2) removes aspirated waste output to the aspiration canister. By means of autonomous rotation, horizontal, and vertical robotic motion control, the treatment probe (114 in FIG. 1) advances through the treatment area while removing tissue according to the 3D treatment plan outline, with coagulation performed simultaneously.

During the entire treatment, the delivery unit (225 in FIG. 2) protects the surrounding tissue from thermal injury and managing hemostasis, while monitoring multiple parameters simultaneously. By way of non-limiting example, these parameters may include temperature, tissue impedance, etc. As part of autonomous motion control of the treatment probe (114 in FIG. 1) distance, location, and velocity is constantly compared to the desired treatment plan outline. The measured locations, depths and rotation angles are all archived, and can be transferred through the internet connection (208 in FIG. 2) for subsequent review.

At treatment completion, the cooling pump (222 in FIG. 2) stops, and the aspiration pump (214 in FIG. 2) removes the remaining aspirated waste output from the treatment area. Finally, the treatment probe (114 in FIG. 1) is retracted to its initial position for removal from the body, and subsequent cleanup and set up for the next treatment case.

### A. Treatment Procedure Summary

Referring again to the overall procedure 400 of FIG. 4, the treatment consists of the following steps:

1. Preparation (sterile set, ultrasound probe, treatment probe—step 410)

2. Insertion/Alignment (lithotomy position, begin imaging, treatment probe, sonogram adjust—step 420)

3. Positioning/Planning (home position, plan development, irrigation—step 430)

4. Treatment (initiation, pulverization, aspiration, hemostasis, motion control—step 440)

5. Cleanup (reports, filtered aspirate, disposal, O.R. reset—step 450)

### B. Results

This invention provides several advantages over existing systems. It improves the precision of tissue treatment, minimizes the risk of collateral damage, and reduces recovery times for patients. Additionally, the integration of cutting, coagulation, and cooling functions into a single system offers a more streamlined and efficient workflow for surgeons. Autonomous operation further reduces the potential for human error, enhancing the overall safety and effectiveness of the procedure.

### VIII. Conclusion

It should be clear that the image-guided autonomous treatment system and method for urological procedures provides a robust and desirable tool to provide enhanced surgical accuracy, reducing the potential for human error, while integrating multiple active treatment interventions to protect surrounding tissues. The novel system and method effectively combines different types of interventions into a single device—for example, autonomous tissue cutting, coagulation, active cooling, intake/output volumetric analysis, with (optional) particulate capture. Treatment plans generated by the system and method can be refined using existing medical data that is processed with advanced computing procedures—such as classical machine learning, ensemble learning or other AI-based approaches.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments of the apparatus and method of the present invention, what has been described herein is merely illustrative of the application of the principles of the present invention. For example, as used herein, the terms “process” and/or “processor” should be taken broadly to include a variety of electronic hardware and/or software based functions and components (and can alternatively be termed functional “modules” or “elements”). Moreover, a depicted process or processor can be combined with other processes and/or processors or divided into various sub-processes or sub-processors. Such sub-processes and/or sub-processors can be variously combined according to embodiments herein. Likewise, it is expressly contemplated that any function, process and/or processor herein can be implemented using electronic hardware, software consisting of a non-transitory computer-readable medium of program instructions, or a combination of hardware and software. Additionally, as used herein various directional and dispositional terms such as “vertical”, “horizontal”, “rotational”, “up”, “down”, “bottom”, “top”, “side”, “front”, “rear”, “left”, “right”, and the like, are used only as relative conventions and not as absolute directions/dispositions with respect to a fixed coordinate space, such as the acting direction of gravity. Additionally, where the term “substantially” or “approximately” is employed with respect to a given measurement, value or characteristic, it refers to a quantity that is within a normal operating range to achieve desired results, but that includes some variability due to inherent inaccuracy and error within the allowed tolerances of the system (e.g., 1-5 percent). Accordingly, this description is meant to be taken only by way of example, and not to otherwise limit the scope of this invention.

What is claimed is:

1. An autonomous system for surgery on a region of a patient comprising:
an imaging transducer that images the region in real time;
a processor that receives the images and recognizes features therein related to the region;
a robotic treatment probe constructed an arranged to perform surgical tissue cutting and coagulation on the region based upon instructions from the processor; and
an active cooling circuit that circulates predetermined volumes of fluid at the region.

2. The system as set forth in claim 1, further comprising an aspiration circuit that directs fluid from the region to a remote location for collection.

3. The system as set forth in claim 1, wherein the aspiration circuit captures fluid and enables volumetric analysis.

4. The system as set forth in claim 3, wherein the aspiration circuit includes a filter that captures particulates for analysis.

5. The system as set forth in claim 3, wherein the region is the peritoneal region and the surgery is a urological procedure thereon.

6. The system as set forth in claim 1, further comprising a graphical user interface constructed and arranged to enable planning of a path of the surgery and treatment probe based upon real-time images overlaid on prior-acquired images from a scanning modality.

7. The system as set forth in claim 6, wherein the imaging transducer comprises an ultrasound transducer that provides images to a user.

8. A method for performing urological surgery with the system of claim 1, comprising the steps of:
preparing the surgical site and locating the imaging transducer and treatment probe with respect to the region;
locating a lithotomy position, beginning imaging with the imaging transducer;
inserting the treatment probe and adjusting a sonogram from the imaging transducer using a user interface;
positioning the treatment probe at a home position, developing a treatment plan on the user interface and irrigating the region; and
performing treatment of the region, including initiation, pulverization, aspiration, hemostasis, based upon motion control of the treatment probe based upon the processor.

9. The method as set forth in claim 8, further comprising providing treatment reports to a user, processing filtered aspirate from the region and disposal of waste.

10. The method as set forth in claim 9, further comprising capturing fluid and performing volumetric analysis.

11. The method as set forth in claim 8, further comprising, operating a graphical user interface for planning of a path of the surgery and treatment probe based upon real-time images overlaid on prior-acquired images from a scanning modality.

12. The method as set forth in claim 11, wherein the images are derived from at least one of ultrasound, X-ray, CT and MRI scans.

13. The method as set forth in claim 8, wherein the imaging transducer is an ultrasound probe

14. The method as set forth in claim 8, wherein the processor is adapted to operate the treatment probe autonomously.

15. The method as set forth in claim 14, wherein the processor employs a 3D treatment plan that maps a treatment region of the patient and follows program steps to perform the method.
